(19) United States
(12) Patent Application Publication
(10) Pub. No.: US 2015/0119723 A1
(43) Pub. Date: Apr. 30, 2015

(54) DIAGNOSTIC PROBE AND INSPECTION APPARATUS COMPRISING SAME

(71) Applicant: GWANGJU INSITUTE OF SCIENCE AND TECHNOLOGY, Gwangju (KR)

(72) Inventors: Jong-Hyun Lee, Gwangju (KR); Hyoung-Ihl Kim, Gimje-si (KR); Gi-Seok Kang, Gwangju (KR); Jae-Cheon Kim, Gwangju (KR)

(21) Appl. No.: 14/399,209

(22) PCT Filed: May 10, 2013
(86) PCT No.: PCT/KR2013/004188

§ 371 Date: Nov. 6, 2014

(30) Foreign Application Priority Data
May 10, 2012 (KR) 10-2012-0049956

(51) Int. Cl. A61B 5/00; A61M 25/00

(52) U.S. Cl. CPC A61B 5/0084; A61M 25/0082
USPC 600/478

(57) ABSTRACT

A diagnostic probe includes a tube having a predetermined length; a deformable cylinder installed by being fitted into the tube such that both ends thereof are exposed to an outside; a guide needle formed in a hollow shape, having a predetermined length, and arranged to surround an outer circumference of the tube; a direction controller connected with an end of the tube, and positioning or vibrating the tube in a radial direction by receiving power from the outside; a vibration generator vibrating the cylinder in an axial direction by receiving power from the outside; and a handle unit connected with the other end of the tube, and connected with the direction controller and the vibration generator.

### BACKGROUND

1. Technical Field

The present disclosure relates to a diagnostic probe and an inspection device having the same, and more particularly, to a diagnostic probe and an inspection device having the same, which are capable of precisely controlling a radial or axial movement of the leading end of the probe, generating a radial or axial vibration, and inspecting cell tissues in the body in real time.

2. Related Art

In general, when an existing disk stimulation test for diagnosing a discogenic pain is performed, a contrast medium is injected through a probe inserted into a disk.

The above-described test that causes a pain by increasing a pressure is difficult to be used for a patient whose disk is severely degenerated, because the contrast medium is likely to leak out of the disk.

Also, the test has a problem in that it cannot stimulate only the micro nerves positioned at a suspected lesion.

Further, when controlling the direction of a conventional probe to be inserted into the body, one end of a steering wire is connected to an end of a tube, and an operator moves an end of the probe by manipulating a handle connected to the other end of the steering wire.

In this regard, however, when the direction of the probe is controlled, a problem may be encountered in that the radial movement of the probe may not be precisely controlled.

A reference document related with the present disclosure is Korean Unexamined Patent Publication No. 10-2010-0119907, in which a technology for simplifying the structure of a probe by providing a piezoelectric vibrator at an end of the probe is disclosed.

### SUMMARY

Various embodiments are directed to a diagnostic probe and an inspection device having the same, which are capable of precisely controlling a radial or axial movement of the leading end of the probe, generating a radial or axial vibration, and inspecting cell tissues in the body in real time.

Also, various embodiments are directed to a diagnostic probe and an inspection device having the same, which are capable of precisely maintaining the movement of the probe in a radial direction desired by an operator, performing a diagnosis by stimulating a lesion portion through vibrating the end of the probe using a motor such as a rotary or linear motor, adjusting the frequency and amplitude of vibrations, and generating vibrations in the radial or axial direction.

In an embodiment, a diagnostic probe may include: a tube having a predetermined length; a deformable cylinder installed by being fitted into the tube such that both ends thereof are exposed to an outside; a guide needle formed in a hollow shape, having a predetermined length, and arranged to surround an outer circumference of the tube; a direction controller connected with an end of the tube, and positioning or vibrating the tube in a radial direction by receiving power from the outside; a vibration generator vibrating the cylinder in an axial direction by receiving power from the outside; and a handle unit connected with the other end of the tube, and connected with the direction controller and the vibration generator.

The direction controller may include a steering wire passing through the guide needle, connected with the tube at one end thereof, and having a predetermined length; and a rotator connected to the other end of the steering wire, and rotated to pull the steering wire.

A plurality of pairs of steering wires may be provided and installed to pass through the guide needle, and each pair of steering wires may be installed to form an angle of 180 degrees with respect to each other.

The rotator may include a rotating handle which is connected with the other end of the steering wire and is rotated to pull the steering wire.

The rotator may include a rotary motor which is connected with the other end of the steering wire and which positions the tube in the radial direction or generates vibrations in a predetermined pattern, by receiving power from the outside.

The vibration generator may include a vibration motor connected to the other end of the cylinder, and vibrating the cylinder in the axial direction; and a controller controlling an operation of the vibration motor, and variably set with a vibration value required for vibration.

The handle unit may include a moving member capable of slidably moving the vibration motor in a lateral direction.

At least one stiffening wire for forming a predetermined retention force may be installed in the guide needle.

The steering wire may include any one of a circular wire and a leaf spring which are formed of a metallic material.

The cylinder may be formed of any one of a metallic wire and an optical fiber.

The optical fiber may include a plastic optical fiber.

In an embodiment, an inspection device may include: the diagnostic probe; an electric sensor installed on an end of the cylinder and measuring impedances of a normal portion and a lesion portion of a disk, or an optical sensor measuring an optical signal; and a storage unit storing a result measured through the cylinder.

### BRIEF DESCRIPTION OF THE DRAWINGS

FIG. 1 is a view illustrating a diagnostic probe in accordance with an embodiment of the present disclosure.

FIG. 2 is a view illustrating a diagnostic probe in accordance with another embodiment of the present disclosure.

FIGS. 3 to 6 are conceptual views of a flexible tube.

FIG. 7 is a diagram illustrating an inspection device having a diagnostic probe in accordance with an embodiment of the present disclosure.

FIG. 8 is a view illustrating that axial vibrations are generated in the diagnostic probe according to the embodiment of the present disclosure.

FIG. 9 is a view illustrating that axial vibrations are generated in the diagnostic probe according to another embodiment of the present disclosure.

FIG. 10 is a view illustrating an example of performing a diagnosis by using the inspection device having the diagnostic probe according to the embodiment of the present disclosure.

### DETAILED DESCRIPTION

Hereafter, a diagnostic probe and an inspection device having the same according to embodiments of the present disclosure will be described with reference to the accompanying drawings.

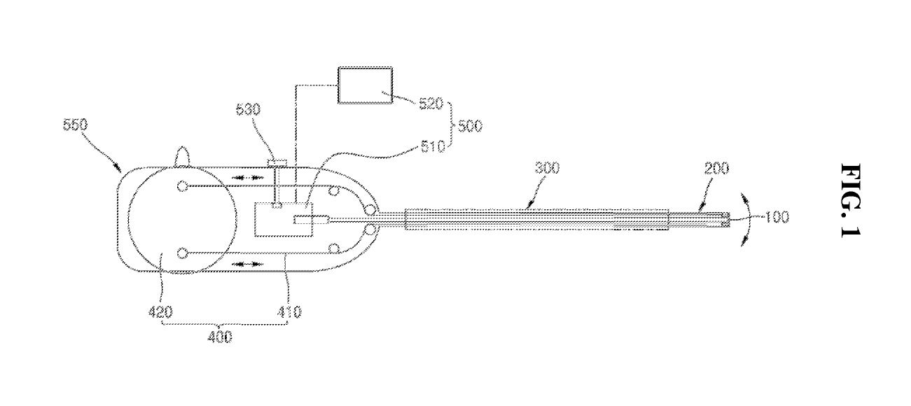

FIG. 1 illustrates a diagnostic probe in accordance with an embodiment of the present disclosure.

Referring to FIG. 1, the diagnostic probe according to the embodiment of the present disclosure may include a tube 200, a cylinder 100, a guide needle 300, a direction controller 400, a vibration generator 500, and a handle unit 550.

The tube 200 has a predetermined length, and is flexibly formed to be capable of bending. The tube 200 is formed in a hollow shape.

The guide needle 300 is formed in a hollow shape. The tube 200 is fitted into the hollow part of the guide needle 300. The guide needle 300 is formed to have a length that is shorter by a predefined length than the length of the tube 200.

The other end of the tube 200 is connected to the handle unit 550.

The cylinder 100 having a predetermined length is inserted into the tube 200. One end of the cylinder 100 is arranged to project from one end of the tube 200 by a preselected length.

The other end of the cylinder 100 is arranged to project from the other end of the tube 200 by a preselected length.

The direction controller 400 serves to position the tube 200 in the radial direction and reciprocatingly move the tube 200.

The direction controller 400 includes a steering wire 410, and a rotator 420 to pull the steering wire 410.

As shown in FIG. 1, a pair of steering wires 410 is provided and is installed to pass through the guide needle 300. The pair of steering wires 410 is installed to maintain 180 degrees with respect to each other.

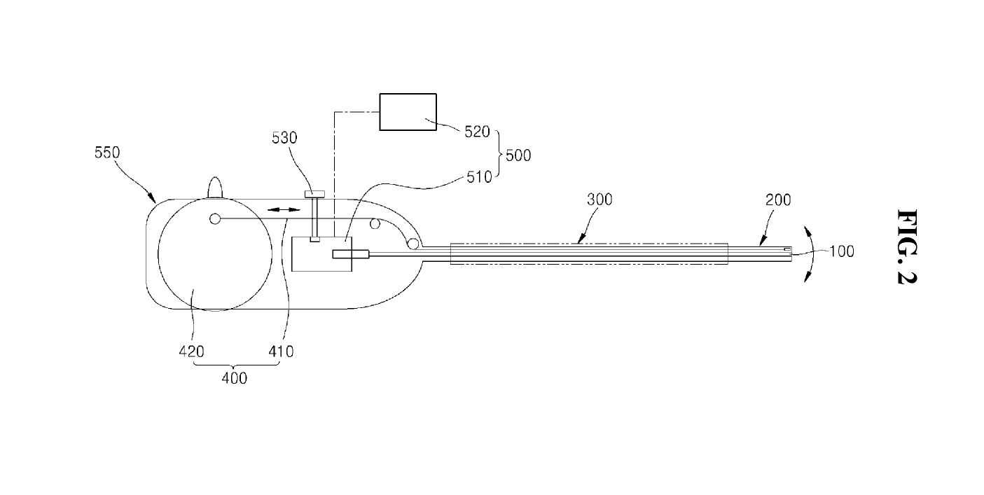

Of course, as shown in FIG. 2, only one steering wire 410 may be provided.

In addition, although not shown in a drawing, a plurality of pairs of steering wires 410 may be provided according to the present disclosure. In this case, each pair of steering wires 410 may be installed in the guide needle 300 to maintain 180 degrees with respect to each other.

One end of the steering wire 410 is connected to the one end of the tube 200. The other end of the steering wire 410 is connected to the rotator 420.

Even when the pair of steering wires 410 is provided, the pair of steering wires 410 is connected in the same manner as described above.

A stiffening wire 310, which induces a predetermined retention force and is capable of being deformed when an external force is applied, is installed in the guide needle 300.

The rotator 420 may be a rotating handle. The rotating handle is rotatably installed at a predetermined position on the handle unit 550.

As the rotator 420 is rotated in one direction or the other direction, the respective steering wires 410 may be pulled or released, and according to this operation, the tube 200 may be moved in the radial direction.

The above-described steering wire 410 may include any one of a circular wire and a leaf spring, which are formed of a metallic material.

In addition, when the rotator 420 is a rotary motor, the rotation shaft of the rotary motor is connected with the other end of each steering wire 410.

The rotary motor may be operated by receiving an electrical signal from an outside, and the one end of the tube 200 may be vibrated in the radial direction according to the operation of the rotary motor.

The rotary motor is electrically connected with a controller 520 which will be described below. The rotary motor may be operated to generate vibrations set by the controller 520.

The vibration generator 500 includes a vibration motor 510 and the controller 520.

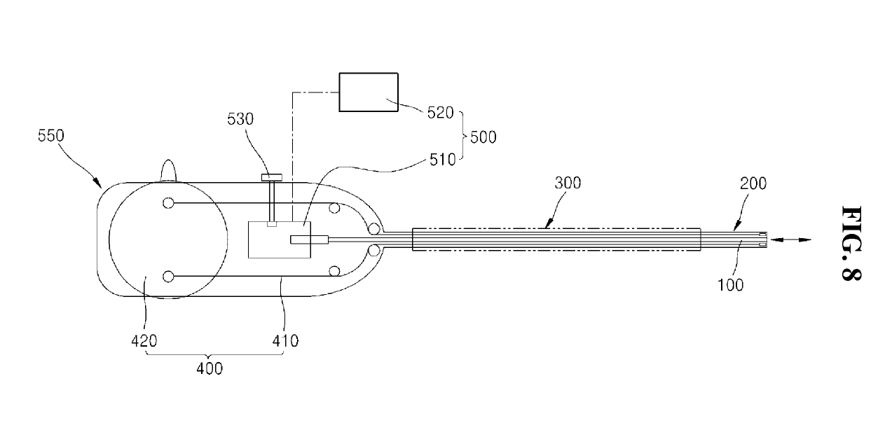

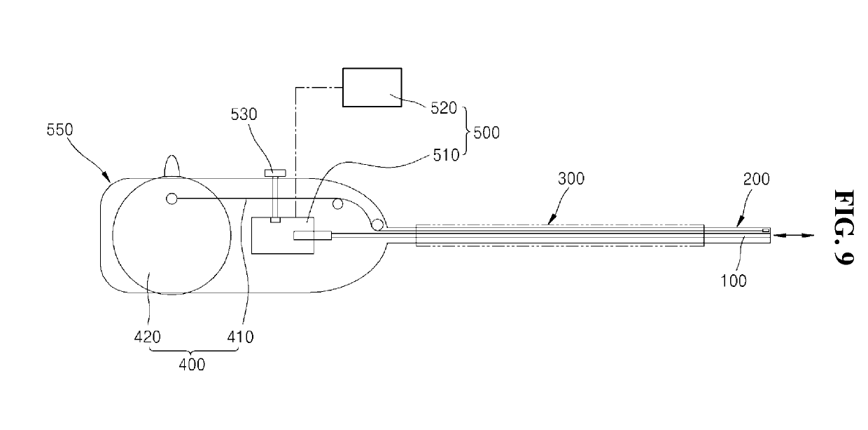

As shown in FIGS. 8 and 9, the vibration motor 510 is installed in the handle unit 550. The vibration motor 510 is connected with the other end of the cylinder 100. The vibration motor 510 receives an electrical signal from the controller 520, and operates the cylinder 100 to vibrate in a predetermined pattern in the axial direction.

Also, a moving member 530 to slidably move the vibration motor 510 in the lateral direction may be additionally provided in the handle unit 550.

The moving member 530 may be a screw type. One end of the moving member 530 is connected to the vibration motor 510, and the other end of the moving member 530 projects out of the handle unit 550. The moving member 530 may be locked to the handle unit 550 in a screw type.

Thus, according to the rotating operation of the moving member 530, the vibration motor 510 may be slidably moved in one direction or the other direction.

The above-described screw type is nothing but a mere example of the moving member 530, and thus, it is to be noted that all techniques for linearly moving the vibration motor 510 may be adopted.

The cylinder 100 according to the embodiment of the present disclosure may be formed of any one of a metallic wire and an optical fiber.

The optical fiber may include a plastic optical fiber.

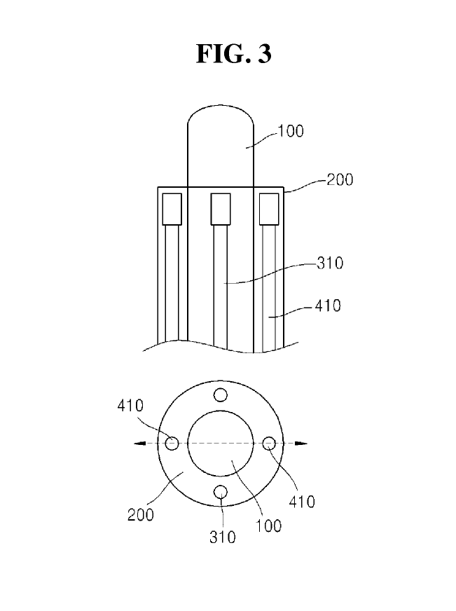

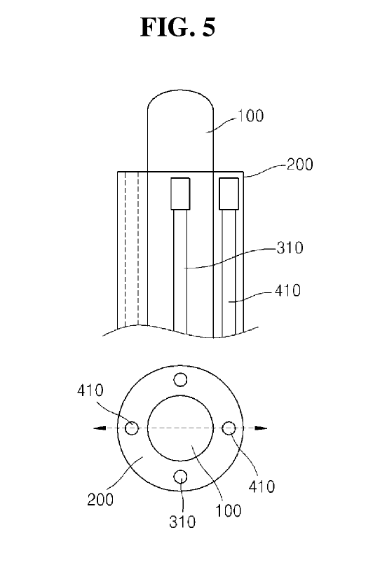

Next, the operation of the diagnostic probe according to the embodiment of the present disclosure will be described with reference to FIGS. 3 to 6.

The tube 200 according to the embodiment of the present disclosure may be formed of a biocompatible polymer-based material. When the tube 200 is steered in the radial direction, two tubes are used. When the steering operation is intended to be controlled with higher precision, four tubes may be used.

[Steering Operation]

Referring to FIGS. 3 to 6, two stiffening wires 310 arranged at 90 degrees with respect to the steering wire 410 are inserted into the tube 200.

One end of each stiffening wire 310 is tightly fixed to an end of the tube 200, and the other end of each stiffening wire 310 is tightly fixed to a predetermined position on the handle unit 550.

Thus, when driving the probe in the radial direction by using the steering wire 410, since the stiffening wires 310 suppresses the movement of the probe in a direction that forms 90 degrees with respect to the driving direction, the end of the probe may be precisely bent in a desired direction.

Thus, it is possible to increase the precision with which an operator places the end of the probe at a desired portion.

[Axial Vibration]

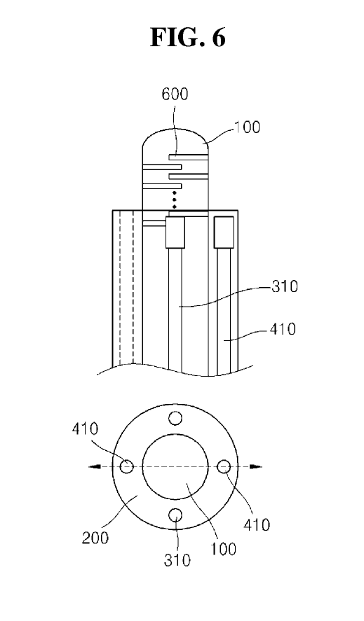

Referring to FIGS. 3 and 6, the vibration motor 510 is connected to the deformable cylinder 100. By controlling the frequency or amplitude of the vibration motor 510, the frequency and magnitude of axial vibration stimuli transmitted to disk tissues through the end of the probe connected to the vibration motor 510 may be changed.

The deformable cylinder 100 uses an optical fiber or desirably a plastic optical fiber.

The cylinder 100 may use a metallic wire.

When the vibration motor 510 is moved away from a center axis by using the moving member 530, since the magnitude of vibrations at the end of the probe is decreased, the magnitude of the axial vibration stimuli transmitted to the disk tissues may be changed more precisely.

The vibration motor 510 may be replaced with a rotary type electric motor which performs a partial reciprocating motion.

[Radial Vibration]

When the rotator 420 connected with the steering wire 410 is replaced with an electric motor, radial vibrations at the end of the probe may be simultaneously realized.

The rotary motor is a linear type or rotary type electric motor, and connects one or two steering wires.

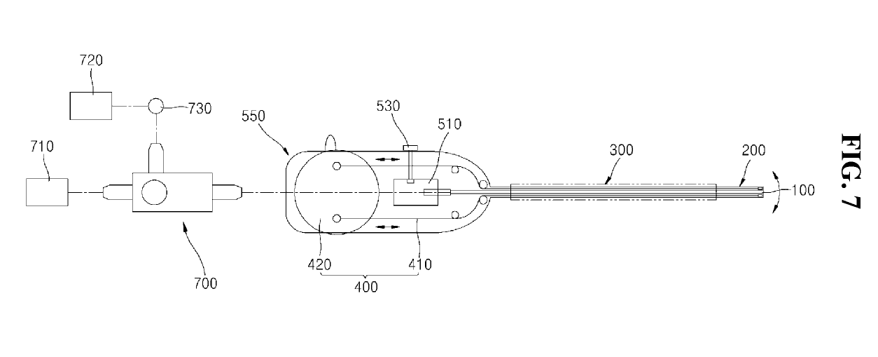

Referring to FIG. 7, although the radial vibrations at the end of the probe may cause a slight damage to the internal tissues of the disk, since the radial vibrations are vibrations in a direction different from the axial vibration, different information may be obtained in view of a medical diagnosis.

Next, an inspection device in accordance with an embodiment of the present disclosure will be described.

Referring to FIG. 7, the inspection device includes an electric sensor 600 which is installed in the end of the cylinder 100 and measures the impedances of the normal portion and the lesion portion of the disk, and a storage unit 700 which stores results measured through the cylinder 100.

The cylinder 100 may be formed of any one of a metallic wire and an optical fiber, and the optical fiber may include a plastic optical fiber.

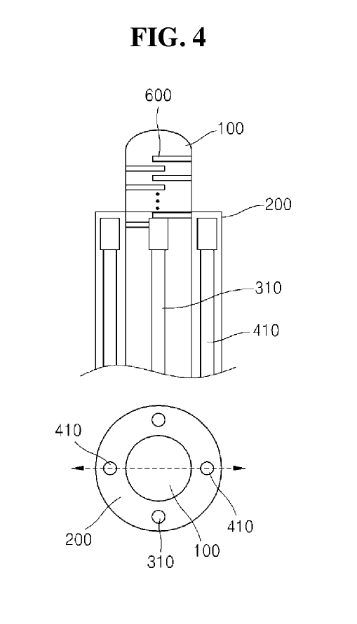

In electrical sensing of FIGS. 4 and 6, the suggested electrode 600 is positioned in the end of the deformable cylinder 100. By measuring the impedance difference between the normal portion and the lesion portion of the disk through using the electrode 600, the precision of a diagnosis may be increased.

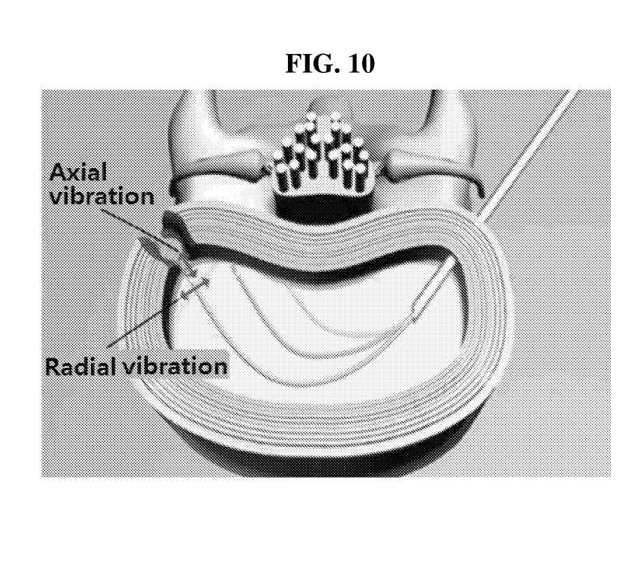

Furthermore, the measurement results are transmitted to the storage unit 700 through the cylinder 100. Referring to FIG. 10, the suggested optical sensing is realized by using an optical fiber or desirably a plastic optical fiber as the deformable cylinder 100. The optical fiber is used as a transmission path for transmitting light.

Thus, a sensing result may be monitored in real time, and, if necessary, inspection information may be stored.

In FIG. 7, the reference numeral 710 designates a laser source, the reference numeral 720 designates a power meter, and the reference numeral 730 designates a detector.

As is apparent from the above descriptions, according to the embodiments of the present disclosure, since precise radial steering may minimize damage to human tissues and a probe may be quickly moved to a suspected lesion of a patient, it is possible to reduce a time required for a diagnosis.

Furthermore, according to the embodiments of the present disclosure, since a cylinder positioned at the end of the probe may be vibrated in a desired direction and with desired frequency and amplitude and an electric motor for vibration is positioned outside a tube (outside the human body), advantages may be provided in terms of design, fabrication and stability.

Moreover, according to the embodiments of the present disclosure, since an electric sensor and an optical sensor using an optical fiber are simultaneously realized, the precision of a diagnosis may be increased.

Also, according to the embodiments of the present disclosure, the real-time monitoring of inspection information may aid an operator to perform a diagnosis, and, by storing the inspection information, the inspection information may be analyzed later as the occasion demands.

While various embodiments have been described above, it will be understood to those skilled in the art that the embodiments described are by way of example only. Accordingly, the disclosure described herein should not be limited based on the described embodiments.

1. A diagnostic probe comprising:
a tube having a predetermined length;
a deformable cylinder installed by being fitted into the tube such that both ends thereof are exposed to an outside;
a guide needle formed in a hollow shape, having a predetermined length, and arranged to surround an outer circumference of the tube;
a direction controller connected with an end of the tube, and positioning or vibrating the tube in a radial direction by receiving power from the outside;
a vibration generator vibrating the cylinder in an axial direction by receiving power from the outside; and
a handle unit connected with the other end of the tube, and connected with the direction controller and the vibration generator.

2. The diagnostic probe of claim 1, wherein the direction controller comprises:
a steering wire passing through the guide needle, connected with the tube at one end thereof, and having a predetermined length; and
a rotator connected to the other end of the steering wire, and rotated to pull the steering wire.

3. The diagnostic probe of claim 2,
wherein a plurality of pairs of steering wires are provided and installed to pass through the guide needle, and
wherein each pair of steering wires are installed to form an angle of 180 degrees with respect to each other.

4. The diagnostic probe of claim 2, wherein the rotator comprises a rotating handle which is connected with the other end of the steering wire and is rotated to pull the steering wire.

5. The diagnostic probe of claim 2, wherein the rotator comprises a rotary motor which is connected with the other end of the steering wire and which positions the tube in the radial direction or generates vibrations in a predetermined pattern, by receiving power from the outside.

6. The diagnostic probe of claim 1, wherein the vibration generator comprises:
a vibration motor connected to the other end of the cylinder, and vibrating the cylinder in the axial direction; and
a controller controlling an operation of the vibration motor, and variably set with a vibration value required for vibration.

7. The diagnostic probe of claim 6, wherein the handle unit comprises a moving member capable of slidably moving the vibration motor in a lateral direction.

8. The diagnostic probe of claim 1, wherein at least one stiffening wire for forming a predetermined retention force is installed in the guide needle.

9. The diagnostic probe of claim 2, wherein the steering wire comprises any one of a circular wire and a leaf spring which are formed of a metallic material.

10. The diagnostic probe of claim 1, wherein the cylinder is formed of any one of a metallic wire and an optical fiber.

11. The diagnostic probe of claim 10, wherein the optical fiber comprises a plastic optical fiber.

12. (canceled)
